(19) United States
(12) Patent Application Publication
(10) Pub. No.: US 2024/0298979 A1
(43) Pub. Date: Sep. 12, 2024

(54) COLLISION AVOIDANCE WHEN POSITIONING A MEDICAL IMAGING DEVICE AND A PATIENT POSITIONING APPARATUS

(71) Applicant: Siemens Healthineers AG, Forchheim (DE)

(72) Inventors: Philipp Roser, Erlangen (DE); Marcus Pfister, Bubenreuth (DE)

(21) Appl. No.: 18/597,909

(22) Filed: Mar. 6, 2024

(30) Foreign Application Priority Data
Mar. 7, 2023 (DE) 10 2023 202 011.4

(51) Int. Cl. A61B 6/10; A61B 6/00; A61B 6/04; A61B 6/46

(52) U.S. Cl. CPC A61B 6/102; A61B 6/0407; A61B 6/4441; A61B 6/461; A61B 6/467; A61B 6/488

(57) ABSTRACT

A method for collision avoidance when positioning a medical imaging device and a patient positioning apparatus includes capturing a representation of an examination object, identifying an examination region based on representation, and identifying an initial acquisition trajectory around an initial isocenter, which enables a 3D reconstruction of the examination region. The imaging device and/or the patient positioning apparatus is positioned such that an isocenter of the medical imaging device coincides with the initial isocenter. A further acquisition trajectory is identified around one further isocenter, which enables a collision-free positioning of the imaging device and a 3D reconstruction of the examination region. The further isocenter specifies a permissible range for relative positionings of the imaging device and the patient positioning apparatus. The imaging device and/or the patient positioning apparatus is repositioned. Repositioning is limited to a permissible range of relative positionings.

This application claims the benefit of German Patent Application No. DE 10 2023 202 011.4, filed on Mar. 7, 2023, which is hereby incorporated by reference in its entirety.

### BACKGROUND

The present embodiments relate to collision avoidance when positioning a medical imaging device and a patient positioning apparatus.

With modern medical imaging devices (e.g., a medical X-ray device and/or an angiography system), not only two-dimensional (2D) fluoroscopy images may be obtained, but also three-dimensional (3D) images of an examination region of an examination object reconstructed based on 2D fluoroscopy images acquired along an acquisition trajectory. These 3D images may map the examination region similar to computer tomography (CT). For example, an arrangement of the X-ray source and the X-ray detector of a C-arm X-ray device may be rotated along the acquisition trajectory around the examination object (e.g., the examination region). In this case, an area penetrated by X-rays (e.g., a volume-of-interest (VOI)) that may be mapped in 3D mapping after reconstruction based on the 2D fluoroscopy images is limited by the size of an X-ray cone. In addition, the VOI is to be mappable for each of the rotation angles. For this purpose, the VOI is to be located in a center of rotation (e.g., an isocenter) of the C-arm X-ray device.

Isocentering of examination regions not arranged centrally with regard to patient geometry (e.g., a border area of the examination object) is often time-consuming, as besides isocentering, a collision-free acquisition trajectory is to be provided. Frequently, isocentering requires repeated acquisition of fluoroscopy images of the examination region in order to bring the examination region into the isocenter by lateral shifting of a patient positioning apparatus. According to this, in the case of a lateral C-arm, freedom from collisions may be checked, as lateral shifting of the patient positioning apparatus and the examination object is severely restricted by the rotating C-arm.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, X-ray dose-efficient and collision-free positioning of a medical imaging device and a patient positioning apparatus is provided for three-dimensional (3D) mapping of an examination region of an examination object.

Independent of the grammatical term usage, individuals with male, female or other gender identities are included within the term.

In a first aspect, the present embodiments relate to a method for collision avoidance when positioning a medical imaging device and a patient positioning apparatus. In doing so, a representation of an examination object that is arranged on a mobile patient positioning apparatus is captured. Further, an examination region of the examination object to be mapped is identified based on the representation. In addition, an initial acquisition trajectory around an initial isocenter is identified. The initial acquisition trajectory enables a 3D reconstruction of the examination region based on image data that may be acquired along the initial acquisition trajectory by the imaging device. Further, the imaging device and/or the patient positioning apparatus are positioned such that an isocenter of the medical imaging device substantially coincides with the initial isocenter. Further, at least one further acquisition trajectory around at least one further isocenter is identified. In this case, the at least one further acquisition trajectory enables collision-free positioning of the imaging device and a 3D reconstruction of the examination region based on image data that may be acquired by the imaging device along the at least one further acquisition trajectory. Further, the at least one further isocenter specifies a permissible range of relative positionings of the imaging device and the patient positioning apparatus. In addition, the imaging device and/or the patient positioning apparatus are repositioned. The repositioning is limited to the permissible range of relative positionings.

Capturing the representation of the examination object may include receiving and/or acquiring the representation of the examination object. Receiving the representation may include, for example, capturing and/or reading out computer-readable memory and/or receiving the representation from a data storage unit (e.g., a database). Further, the representation of a processing unit of a medical imaging device may be provided. According to one embodiment, the representation may include initial (e.g., pre-captured) medical image data of the examination object. For example, initial medical image data may be acquired as the representation of the examination object by the medical imaging device. Alternatively or in addition, the representation may include a model (e.g., a volume model, such as a volume network model, or a skeletonized model) of the examination object. For example, a statistical and/or standardized model may be adapted to the examination object based on parameters of the examination object. Alternatively or in addition, the model may have been provided based on pre-captured data (e.g., initial image data) and/or segmentation (e.g., semi-automatic or automatic segmentation) of the initial image data of the examination object. For example, the model may be configured as a digital twin of the examination object.

The examination object may be, for example, a female human and/or animal patient and/or a male human and/or animal patient and/or an examination phantom. The examination region of the examination object may, for example, include a spatial section (e.g., a volume) of the examination object that has an anatomical object to be mapped (e.g., an organ and/or a tissue) and/or a medical object. The patient positioning apparatus may, for example, include a patient couch and/or a patient table and/or a patient chair. Further, the patient positioning apparatus may be mobile (e.g., translatable and/or rotatable and/or movable and/or tiltable). As a result, the examination object arranged on the patient positioning apparatus may be repositionable.

The medical imaging device may include a mobile medical X-ray device (e.g., a medical C-arm X-ray device and/or a cone-beam CT (CBCT)), and/or a mobile computer tomography system (CT system), and/or a mobile magnetic resonance imaging system (MRI system), and/or a mobile positron emission tomography system (PET system), and/or a mobile ultrasound device.

The identification of the examination region to be mapped may include segmenting of an image and/or a model of the anatomical and/or medical object in the representation. Alternatively or in addition, the examination region to be mapped may be identified based on a user input (e.g., by annotation). In one embodiment, the identification of the examination region to be mapped may include determining spatial positioning (e.g., a spatial position and/or orientation and/or pose) and/or a spatial extent, for example, of a volume of the anatomical and/or medical object to be mapped in the examination object based on the representation. The representation of the examination object may be registered with a coordinate system of the examination object and/or the imaging device and/or the patient positioning apparatus.

The initial isocenter may be identified manually, semi-automatically, or fully automatically. According to a first variant, the initial isocenter may be manually identified (e.g., specified) based on a user input by operating personnel. For example, the user input may specify spatial coordinates (e.g., a point) for the initial isocenter (e.g., in a coordinate system of the imaging device and/or the patient positioning apparatus). According to a second variant, the initial isocenter may be identified semi-automatically. For example, the examination region to be mapped (e.g., an anatomical structure to be mapped and/or a medical object to be mapped) may be identified manually based on a user input by the operating personnel with regard to the representation of the examination object. Further, the initial isocenter may be identified based on geometric and/or anatomical features of the examination region to be mapped (e.g., the anatomical structure to be mapped and/or the medical object to be mapped) that are mapped and/or modeled in the representation. According to a third variant, the initial isocenter may be identified fully automatically, for example, based on anatomical and/or geometric features of the examination region to be mapped, which are mapped and/or modeled in the representation.

The initial acquisition trajectory may predefine a predefined (e.g., standardized) movement trajectory for the imaging device around the initial isocenter. The initial acquisition trajectory may, for example, extend essentially in a plane through the initial isocenter and at a predefined angle (e.g., a predefined orientation) with regard to the examination object (e.g., with regard to a longitudinal axis of the examination object). In one embodiment, the initial acquisition trajectory may be predefined in a predefined positional relationship with regard to the examination object. For example, the initial acquisition trajectory may extend in a plane through the initial isocenter and in essentially vertical alignment with regard to the longitudinal axis of the examination object. The initial acquisition trajectory may be identified (e.g., without taking into account possible collisions with the patient positioning apparatus and/or the examination object). Thus, although the initial acquisition trajectory may enable the 3D reconstruction of the examination region, the initial acquisition trajectory may specify a non-collision-free positioning of the imaging device. In one embodiment, the initial acquisition trajectory and the initial isocenter may be identified such that the examination region to be mapped (e.g., in full and/or truncation-free) is arranged within a volume of the imaging device that may be reconstructed when positioned along the initial acquisition trajectory.

In one embodiment, the medical imaging device may be positioned with regard to the examination object (e.g., the patient positioning apparatus), such that the isocenter of the medical imaging device substantially coincides with the initial isocenter. Alternatively or in addition, the patient positioning apparatus (e.g., with the examination object arranged thereon) may be positioned in such a way with regard to the medical imaging device that the isocenter of the medical imaging device substantially coincides with the initial isocenter. Alternatively or in addition, the medical imaging device and the patient positioning apparatus may be positioned relative to one another such that the isocenter of the medical imaging device substantially coincides with the initial isocenter. The positioning of the medical imaging device and/or the patient positioning apparatus may include moving (e.g., manually or semi-automatically or automatically, such as translating and/or rotating and/or tilting) the medical imaging device and/or the patient positioning apparatus. This initial positioning of the imaging device and/or the patient positioning apparatus may take place without collision.

The identification of the at least one further acquisition trajectory around the at least one further isocenter may include identification of a further acquisition trajectory around one further isocenter or identification of a plurality of further acquisition trajectories around one further isocenter in each case. In one embodiment, the at least one further acquisition trajectory and the at least one further isocenter may be determined such that the imaging device may be positioned along the at least one further acquisition trajectory in a collision-free manner and the examination region to be mapped may be reconstructed in 3D based on image data that may be acquired by the imaging device along the at least one further acquisition trajectory. In one embodiment, the identification of the at least one further acquisition trajectory around the at least one further isocenter may include a simulation of the positioning of the imaging device along the at least one further acquisition trajectory and the patient positioning apparatus with the examination object arranged thereon in order to exclude a collision.

The 3D reconstructability of the examination region to be mapped may be limited by an imaging geometry of the imaging device (e.g., a geometry of an X-ray source and an X-ray-sensitive surface of an X-ray detector). In one embodiment, the at least one further acquisition trajectory and the at least one further isocenter may be identified such that the examination region to be mapped is arranged (e.g., in full and/or truncation-free) within a reconstructable volume of the imaging device when positioned along the at least one further acquisition trajectory. In addition, a spatial safety margin may be specified with regard to a border area of the reconstructable volume. In this case, the at least one further acquisition trajectory and the at least one further isocenter may be identified such that the examination volume within the reconstructable volume is arranged in a reduced manner around the safety margin. This may provide the (e.g., truncation-free) 3D reconstructability of the examination region to be mapped based on image data that may be acquired by the imaging device along the at least one further acquisition trajectory. In one embodiment, the at least one further acquisition trajectory may specify a plurality of positionings (e.g., spatial positions and/or orientations and/or poses and/or angulations) for the medical imaging device. As a result of the image data that may be acquired by the imaging device along the at least one further acquisition trajectory (e.g., in the plurality of positions), the examination region to be mapped may be mapped from different directions (e.g., angulations). This enables the 3D reconstructability of the examination region to be mapped based on the image data that may be acquired.

The at least one further acquisition trajectory may be identified (e.g., with regard to its completeness) by a method for optimizing acquisition trajectories. Such optimization methods are known, for example, from the publications of S. Hatamikia et al., “Source-detector trajectory optimization in cone-beam computed tomography: a comprehensive review on today's state-of-the-art,” Phys. Med. Biol., 2022, 67, 16TR03 and G. Herl et al., “Scanning trajectory optimisation using a quantitative Tuy-based local quality estimation for robot-based X-ray computed tomography,” Nondestructive Testing and Evaluation, 2020, 35:3, 287-303.

The at least one further isocenter (e.g., the plurality of further isocenters) may specify the permissible range of relative positionings of the imaging device and the patient positioning apparatus.

In one embodiment, the imaging device and/or the patient positioning apparatus may be repositioned relative to one another (e.g., for fine adjustment of the relative positioning). The medical imaging device may be repositioned relative to the examination object (e.g., the patient positioning apparatus). Alternatively or in addition, the patient positioning apparatus (e.g., with the examination object arranged thereon) may be repositioned relative to the medical imaging device in this way. Alternatively or in addition, the medical imaging device and the patient positioning apparatus may be repositioned relative to one another. The repositioning of the medical imaging device and/or the patient positioning apparatus may include moving (e.g., manually or semi-automatically or automatically, such as translating and/or rotating and/or tilting) the medical imaging device and/or the patient positioning apparatus.

The repositioning (e.g., degrees of freedom of movement of the imaging device and/or the patient positioning apparatus) may be limited (e.g., mechanically and/or electromagnetically and/or pneumatically) to the permissible range of relative positionings. In one embodiment, the initial and the at least one isocenter (e.g., additionally the at least one further acquisition trajectory) may each be identified with regard to the patient positioning apparatus (e.g., in a common and/or registered coordinate system of the imaging device and the patient positioning apparatus). Further, the at least one further isocenter may define a spatial area within which the permissible relative positionings of the medical imaging device and the patient positioning apparatus are arranged.

Limiting the repositioning to the permissible range of relative positionings may be assisted, for example, by the action of a counterforce that opposes a force for repositioning the imaging device and/or the patient positioning apparatus. For example, the counterforce may be provided by mechanical and/or electromagnetic and/or pneumatic interaction of a braking apparatus. The provision of the counterforce may be provided depending on an approach of the imaging device and/or the patient positioning apparatus to a border area of the permissible range. Alternatively or in addition, repositioning may be limited by adjusting (e.g., limiting) a control system (e.g., of degrees of freedom for controlling, the imaging device, and/or the patient positioning apparatus).

In one embodiment, the acts of identifying the at least one further acquisition trajectory and repositioning the imaging device and/or the patient positioning apparatus (e.g., selectively) are only carried out if the initial acquisition trajectory specifies a non-collision-free positioning of the imaging device.

The embodiment may enable X-ray dose-efficient and collision-free positioning of the medical imaging device and the patient positioning apparatus for collision-free 3D mapping of the examination region to be mapped. In addition, the positioning of the medical imaging device and the patient positioning apparatus for collision-free 3D mapping of the examination region to be mapped may be carried out in a particularly time-efficient manner using the proposed method.

In a further embodiment of the method, a plurality of further acquisition trajectories around one further isocenter in each case may be identified. The further acquisition trajectories in each case enable the collision-free positioning of the imaging device and the 3D reconstruction of the examination region based on image data that may be acquired by the imaging device along the respective further acquisition trajectory.

In one embodiment, all further acquisition trajectories around one further isocenter in each case may be identified. The further acquisition trajectories enable the collision-free positioning of the imaging device and the 3D reconstruction of the examination region based on image data that may be acquired by the imaging device along the respective further acquisition trajectory. The identification of the further acquisition trajectories and the associated further isocenters in each case may first include the identification of potential acquisition trajectories (e.g., all potential, acquisition trajectories) that enable the 3D reconstruction of the examination region based on image data that may be acquired by the imaging device along the respective potential acquisition trajectory. The criteria described above may be used to enable the 3D reconstruction of the examination region to be mapped. Further, the acquisition trajectories may be identified from the set of potential acquisition trajectories as the further acquisition trajectories that enable a collision-free positioning of the imaging device. The identification of the further acquisition trajectories from the set of potential acquisition trajectories may include a simulation of the positioning of the imaging device along the potential acquisition trajectories and the patient positioning apparatus with the examination object arranged thereon in order to exclude a collision.

The plurality of further isocenters may specify (e.g., span) the permissible range of relative positionings of the imaging device and the patient positioning apparatus.

The embodiment may enable the imaging device and/or the patient positioning apparatus to be repositionable according to the degrees of freedom predefined by the permissible range.

In a further embodiment of the method, when repositioning the imaging device and/or the patient positioning apparatus, a speed of movement of the imaging device and/or the patient positioning apparatus may be reduced when approaching a border area of the permissible range of relative positionings.

In one embodiment, repositioning of the imaging device and/or the patient positioning apparatus may include movement of the imaging device and/or the patient positioning apparatus (e.g., a relative movement of the imaging device and the patient positioning apparatus, such as a translation and/or rotation). In this case, a speed of movement of the imaging device and/or the patient positioning apparatus (e.g., a relative speed of movement between the imaging device and the patient positioning apparatus) may be reduced when approaching a border area of the permissible range of relative positionings (e.g., to a standstill on reaching the border area). In one embodiment, the speed of movement may take place as a result of the provision (e.g., the action) of the counterforce (e.g., counter to a direction of movement, such as a direction of translation and/or direction of rotation, of the imaging device and/or the patient positioning apparatus). The embodiment may prevent leaving the permissible range. In addition, as a result of this, collisions of the imaging device and/or the patient positioning apparatus and/or the examination object may be avoided. In addition, the operating personnel may approach the border area by reducing the speed of movement of the imaging device and/or the patient positioning apparatus (e.g., haptically). In addition, as a result of the reduced speed of movement, the accuracy of repositioning the imaging device and/or patient positioning apparatus may be improved at or in the border area.

In a further embodiment of the method, a user input by operating personnel may be captured by an input unit. The repositioning of the imaging device and/or the patient positioning apparatus may be carried out depending on the user input.

The input unit may include, for example, a keyboard and/or a button and/or a joystick and/or a touchpad and/or a microphone (e.g., for speech recognition), and/or a camera (e.g., for gesture capture). In one embodiment, the input unit may be integrated into a display unit (e.g., as a resistive and/or capacitive touchscreen). The input unit may be arranged on the imaging device and/or the patient positioning apparatus (e.g., at least partially integrated into the imaging device and/or the patient positioning apparatus). Alternatively, the input unit may be arranged at a distance from the imaging device and/or the patient positioning apparatus.

The user input may specify target positioning (e.g., target relative positioning) for the imaging device with regard to the patient positioning apparatus. Target positioning may specify a spatial target position (e.g., a target position for the isocenter of the imaging device) and/or a targeting and/or a target pose for the imaging device. Alternatively or in addition, the user input may specify a movement parameter (e.g., a speed of movement and/or movement direction and/or movement trajectory) for repositioning of the imaging device and/or the patient positioning apparatus.

The input unit may capture the user input of the operating personnel and provide a corresponding signal (e.g., a control signal) to a processing unit, the imaging device, and/or the patient positioning apparatus. The processing unit may control (e.g., coordinate) the repositioning (e.g., a movement and/or relative movement) of the imaging device and/or the patient positioning apparatus as a function of the control signal. The processing unit may provide a corresponding signal to the imaging device and/or the patient positioning apparatus for this. Alternatively or in addition, the repositioning of the imaging device and/or the patient positioning apparatus may be controllable (e.g., directly) by the control signal.

The embodiment may enable safe control of repositioning by the operating personnel by limiting repositioning to the permissible range.

In a further embodiment of the method, a graphic display of the permissible range may be displayed by a display unit.

The display unit may include a monitor and/or a projector and/or a display and/or a pair of data glasses. In one embodiment, a graphic display of the permissible range of relative positionings may be displayed by the display unit. The graphic display of the permissible range may include a graphic display of the initial and at least one further isocenter. Alternatively or in addition, the graphic display of the permissible range may include a graphic display of the degrees of freedom of movement of the imaging device and/or the patient positioning apparatus (e.g., a graphic display of the border area and/or a spatial limitation of the permissible range). In one embodiment, in addition, a graphic display of a current positioning of the imaging device and/or the patient positioning apparatus (e.g., a current relative positioning of the imaging device and the patient positioning apparatus) with regard to the graphic display of the permissible range may be displayed by the display unit (e.g., integrated and/or superimposed). The current positioning of the imaging device and/or the patient positioning apparatus may be provided by the imaging device and/or the patient positioning apparatus and/or by a capture unit for capturing the current positioning (e.g., a camera system). The display unit may display the graphic display of the permissible range on a display area and/or as augmented and/or virtual reality.

In one embodiment, the user input by the operating personnel may be captured by the input unit with regard to the graphic display of the permissible range.

The embodiment may support operating personnel in the repositioning of the imaging device and/or the patient positioning apparatus within the permissible range.

In a further embodiment of the method, the representation of the examination object may include an image and/or a model of at least one anatomical structure and/or a medical object in the examination object. In this case, a spatial area that includes the anatomical structure and/or the medical object at least partially (e.g., in full) may be identified as the examination region to be mapped.

The at least one anatomical structure may include an organ (e.g., a hollow organ, such as an artery and/or a vein) and/or a tissue and/or a tumor. Further, the at least one medical object may include a surgical and/or a diagnostic instrument (e.g., a catheter) and/or an implant (e.g., a stent). The at least one anatomical structure (e.g., a plurality of anatomical structures) and/or the at least one medical object (e.g., a plurality of medical objects) may be arranged in the examination object. Further, the plurality of anatomical and/or medical objects may be mapped and/or modeled in the representation.

The representation of the examination object may include an image (e.g., initial, pre-captured medical image data) of the at least one anatomical structure and/or of the medical object in the examination object. Alternatively or in addition, the representation of the examination object may include a model (e.g., a volume model, such as a volume network model) or a skeletonized model (e.g., a central line model) of the at least one anatomical structure and/or the at least one medical object.

In one embodiment, the spatial area (e.g., a spatial volume including the at least one anatomical structure and/or the at least one medical object) at least partially (e.g., in full) may be identified as the examination region to be mapped. The embodiment may enable a particularly intuitive and X-ray dose-efficient identification of the examination region to be mapped.

In a further embodiment of the method, the representation may in each case have a candidate position with regard to the mapped and/or modeled anatomical structures and/or the medical object. The initial isocenter may be identified at a candidate position within the examination region to be mapped.

The representation (e.g., the image and/or the model) of the examination object may in each case have a candidate position with regard to the at least one mapped anatomical structure (e.g., the plurality of mapped anatomical structures) and/or the at least one mapped medical object (e.g., the plurality of mapped medical objects). For example, the representation may have the at least one candidate position (e.g., a plurality of candidate positions) as annotations with regard to the mapped and/or modeled at least one anatomical structure and/or with regard to the mapped and/or modeled at least one medical object. The candidate positions and/or the representation of the examination object may be acquired using a coordinate system of the imaging device and/or the patient positioning apparatus. The candidate positions may be specified, for example, based on geometric and/or anatomical features of the least one anatomical structure and/or the at least one medical object. For example, the candidate position may mark a geometric center and/or a focal point (e.g., an organ focal point) and/or a reference point (e.g., a marker position and/or a landmark) of the at least one anatomical structure and/or the at least one medical object.

In one embodiment, the initial isocenter may be identified (e.g., defined) at a candidate position within the examination region to be mapped. If the examination region to be mapped includes only one anatomical structure to be mapped or only one medical object to be mapped, the initial isocenter may be identified at the candidate position of the anatomical structure to be mapped or of the medical object to be mapped. If the examination region to be mapped includes a plurality of anatomical structures and/or medical objects to be mapped, the initial isocenter may be identified at one of the plurality of candidate positions. In one embodiment, the candidate position may be selected from the plurality of candidate positions that enables a 3D reconstruction of all the anatomical structures and/or medical objects to be mapped and a collision-free positioning of the imaging device. The initial isocenters may be selected from the plurality of candidate positions (e.g., manually, such as based on a user input, or automatically, such as using an optimization algorithm).

The embodiment may enable particularly efficient initial isocentering.

In a further embodiment of the method, the initial isocenter may be identified based on a user input by operating personnel using an input unit.

In one embodiment, a user input by the operating personnel may be captured by the input unit. According to a first variant, the user input may have a specification for at least one anatomical structure to be mapped and/or for at least one medical object to be mapped. For example, a graphic display of the representation of the examination object may be displayed by the display unit. In this case, the user input by the operating personnel may be captured with regard to the graphic display of the representation. Based on the user input, the at least one anatomical structure to be mapped and/or the at least one anatomical object to be mapped may be identified. According to a further variant, the user input may have a specification for a positioning of the initial isocenter. The user input may specify the initial isocenter, for example, in coordinates of the coordinate system of the imaging device and/or the patient positioning apparatus. Alternatively, the user input may specify the initial isocenter with regard to the graphic display of the representation of the examination object that is acquired with the coordinate system of the imaging device and/or the patient positioning apparatus (e.g., as one of the candidate positions).

The embodiment may enable initial isocentering by the operating personnel.

In a further embodiment of the method, the initial isocenter and/or the at least one further isocenter may be identified based on geometric and/or anatomical features of the examination region to be mapped.

The geometric features may include, for example, a contour and/or a contrast and/or edges and/or a line and/or a marker structure that are mapped or modeled in the representation of the examination region. Further, anatomical features may include, for example, anatomical landmarks and/or a tissue boundary.

The initial isocenter may be identified semi-automatically or fully automatically based on the geometric and/or anatomical features of the examination region to be mapped. For example, the initial isocenter may be identified semi-automatically by manually identifying the examination region to be mapped (e.g., the anatomical structure to be mapped and/or the medical object to be mapped) based on user input by the operating personnel with regard to the representation of the examination object of the examination region to be mapped, and the initial isocenter is identified based on the geometric and/or anatomical features of the examination region to be mapped (e.g., the anatomical structure to be mapped and/or the medical object to be mapped), which are mapped and/or modeled in the representation. Alternatively, the initial isocenter may be identified fully automatically based on the anatomical and/or geometric features of the examination region to be mapped, which are mapped and/or modeled in the representation.

In one embodiment, a geometric center and/or a focal point (e.g., an organ focal point) and/or a reference point (e.g., a marker position and/or a landmark) of the examination region to be mapped (e.g., the at least one anatomical structure and/or the at least one medical object) may be determined based on geometric and/or anatomical features of the examination region to be mapped. The initial isocenter may be identified at the geometric center and/or focal point and/or reference point of the examination region to be mapped.

In one embodiment, a spatial extent (e.g., a tissue boundary and/or a volume and/or a surface) of the examination region to be mapped may be determined based on geometric and/or anatomical features. Based on the spatial extent of the examination region to be mapped, the at least one further acquisition trajectory and the at least one further isocenter (e.g., a plurality of acquisition trajectories around one further isocenter in each case) may be identified such that the volume to be mapped may be reconstructed (e.g., completely) based on 3D image data that may be acquired along the at least one further acquisition trajectory by the imaging device, and the imaging device may be positioned collision-free along the at least one further acquisition trajectory.

In a further embodiment of the method, the medical imaging device may include a medical X-ray device having an X-ray detector and an X-ray source that are arranged in a defined arrangement on an arcuate structure. The initial and the at least one further acquisition trajectory may specify a movement of the defined arrangement around the respective isocenter.

The X-ray source and the X-ray detector may be arranged (e.g., fixed) in a defined arrangement to the arcuate (e.g., circular arc-shaped) structure (e.g., a gantry and/or a C-arm). The X-ray source may be configured to emit X-rays to illuminate the examination object. Further, the X-ray detector may be configured to detect the X-rays emitted by the X-ray source and to provide a signal as a function of the detected X-rays. In one embodiment, the X-ray source and the X-ray detector may be arranged opposite one another on the arcuate structure. The X-ray radiation emitted by the X-ray source illuminates an X-ray-sensitive surface of the X-ray detector (e.g., after an interaction with the examination object).

The initial and the at least one further acquisition trajectory (e.g., the plurality of further acquisition trajectories) may each specify a movement of the defined arrangement of the X-ray source and the X-ray detector around the respective isocenter (e.g., the initial and the at least one further isocenter). For example, the initial and the at least one further acquisition trajectory may in each case specify a movement (e.g., a translation and/or a rotation) of the X-ray source and the X-ray detector in the defined arrangement around the respective isocenter.

The initial and the at least one further isocenter may form a center of rotation of the respective acquisition trajectory (e.g., of the initial and the at least one further acquisition trajectory). In addition, a central beam of the X-ray radiation emittable by the X-ray source may pass through the respective isocenter in an arrangement along the initial and the at least one further acquisition trajectory.

The embodiment may enable an X-ray dose-efficient positioning of the medical imaging device and the patient positioning apparatus for collision-free 3D mapping of the examination region to be mapped based on image data that may be acquired by the medical X-ray device.

In a further embodiment of the method, image data of the examination region may be acquired by the imaging device after the repositioning of the imaging device. According to this, a result data set having a 3D image of the examination region may be reconstructed and provided from the image data.

In one embodiment, image data (e.g., projection images) of the examination object may be acquired by the imaging device after the repositioning of the imaging device with regard to the patient positioning apparatus, (e.g., with regard to the examination object). The isocenter of the imaging device may be arranged within the permissible range after repositioning at a final isocenter (e.g., the initial or a further isocenter of the at least one further isocenter). In one embodiment, the imaging device for acquiring the image data may be arranged (e.g., moved) along a final acquisition trajectory (e.g., the initial or a further acquisition trajectory of the at least one further acquisition trajectory). The final acquisition trajectory corresponds to the final isocenter. In an embodiment of the imaging device as a medical X-ray device (e.g., a medical C-arm X-ray device), the isocenter of the X-ray device may be arranged at the final isocenter after repositioning. Further, projection images (e.g., X-ray projection images) of the examination object may be acquired by the X-ray device. In this case, the defined arrangement of the X-ray source and the X-ray detector may be moved (e.g., rotated and/or translated) along the final acquisition trajectory. Further, the X-ray source may emit X-rays at predefined acquisition positions along the acquisition trajectory (e.g., at predefined angulations with regard to the examination object) to illuminate the examination object. The X-ray detector may detect the X-ray radiation after an interaction with the examination object and provide a corresponding signal.

Further, the result data set having the 3D image of the examination region may be reconstructed from the image data (e.g., the projection images), for example, using a filtered rear projection.

The provision of the result data set may include storage on a computer-readable storage facility medium and/or display on a display unit and/or transmission to a processing unit. For example, a graphic display of the result data set may be displayed by the display unit.

The embodiment can enable collision-free 3D mapping of the examination region to be mapped.

In a second aspect, the present embodiments relate to a system including a medical imaging device and a patient positioning apparatus. The system is configured to carry out a method for collision avoidance when positioning a medical imaging device and a patient positioning apparatus.

The advantages of the system essentially correspond to the advantages of the method for positioning a medical imaging device and a patient positioning apparatus. Features, advantages, or alternative embodiments mentioned here may likewise also be transferred to the other claimed objects and vice versa.

In a third aspect, the present embodiments relate to a computer program that may be loaded directly into a storage facility of a processing unit, having program sections, to perform all the acts of a proposed method for collision avoidance when positioning a medical imaging device and a patient positioning apparatus when the program sections are executed by the processing unit. The computer program product may include software with a source code that still needs to be compiled and linked or only interpreted, or an executable software code that only needs to be loaded into the processing unit for execution. Rapid, identically repeatable, and robust performance of the method for collision avoidance is possible using the computer program product when positioning an imaging device and a patient positioning apparatus. The computer program product is configured such that the computer program product may perform the method acts according to the present embodiments using the processing unit. In addition to the computer program, such a computer program product may optionally include additional components, such as, for example, documentation and/or additional components, as well as hardware components, such as, for example, hardware keys (e.g., dongles, etc.) for using the software.

The advantages of the computer program product essentially correspond to the advantages of the method for collision avoidance when positioning a medical imaging device and a patient positioning apparatus. Features, advantages, or alternative embodiments mentioned here may likewise also be transferred to the other claimed objects and vice versa.

The present embodiments may also be based on a computer-readable storage facility medium and/or electronically readable data carrier on which program sections that may be read and executed by a processing unit are stored in order to carry out all the acts of the method for collision avoidance when positioning a medical imaging device and a patient positioning apparatus when the program sections are executed by the processing unit. A largely software-based implementation has the advantage that processing units already used in the past may also be simply retrofitted by a software update in order to work in the manner according to the present embodiments.

### BRIEF DESCRIPTION OF THE DRAWINGS

Example embodiments of the invention are shown in the drawings and are described in more detail hereinafter. In different figures, the same reference characters are used for the same features. The diagrams show:

FIGS. 1 and 2 are diagrammatic views of various embodiments of a method for collision avoidance when positioning a medical imaging device and a patient positioning apparatus;

FIG. 3 is a diagrammatic view of an embodiment of a system;

FIG. 4 is a diagrammatic view of an example of repositioning of the medical imaging device and the patient positioning apparatus from various viewing perspectives; and

FIG. 5 is a diagrammatic view of a representation of an examination object.

### DETAILED DESCRIPTION

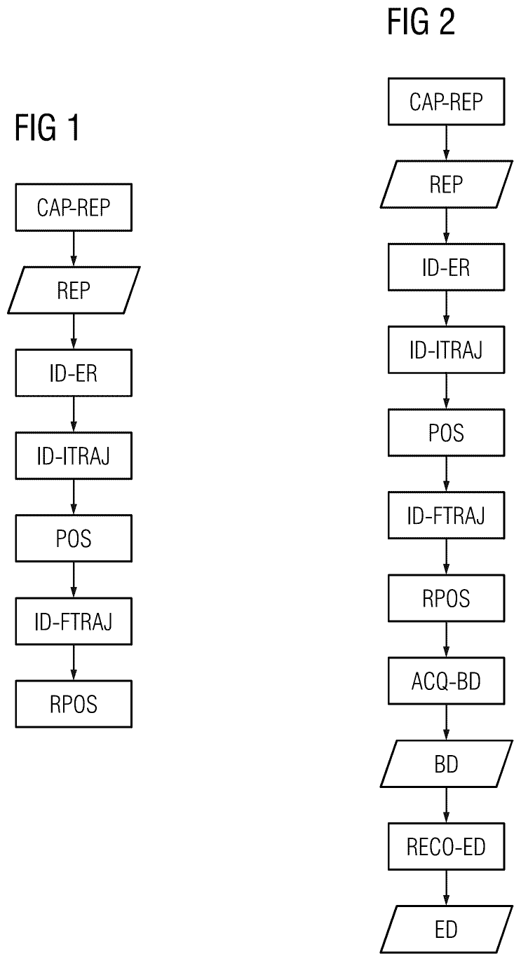

FIG. 1 shows a diagrammatic view of an embodiment of a method for collision avoidance when positioning an imaging device and a patient positioning apparatus. In this case, a representation REP of an examination object that is arranged on a mobile patient positioning apparatus may be captured CAP-REP. Further, an examination region of the examination object to be mapped may be identified based on the representation REP, ID-REP. In addition, an initial acquisition trajectory around an initial isocenter may be identified ID-ITRAJ. Further, the medical imaging device and/or the patient positioning apparatus may be positioned in such a way, POS, that an isocenter of the medical imaging device substantially coincides with the initial isocenter. In addition, at least one further acquisition trajectory around at least one further isocenter may be identified ID-FTRAJ. In this case, the initial and the at least one further acquisition trajectory may enable collision-free positioning of the imaging device and a 3D reconstruction of the examination region based on image data that may be acquired along the respective acquisition trajectory by the imaging device. In addition, the initial and the at least one further isocenter may specify a permissible range of relative positionings of the imaging device and the patient positioning apparatus. Further, the imaging device and/or the patient positioning apparatus may be repositioned RPOS. In this case, repositioning RPOS may be limited to the permissible range of relative positionings.

In one embodiment, a plurality of further acquisition trajectories may be identified around a respective further isocenter ID-FTRAJ. The further acquisition trajectories each enable the collision-free positioning of the imaging device and the 3D reconstruction of the examination region based on image data that may be acquired by the imaging device along the respective further acquisition trajectory.

In one embodiment, when repositioning RPOS the imaging device and/or the patient positioning apparatus, a speed of movement of the imaging device and/or the patient positioning apparatus may be reduced when approaching a border area of the permissible range of relative positionings.

In one embodiment, a user input by operating personnel may be captured by an input unit. In this case, the repositioning RPOS of the imaging device and/or the patient positioning apparatus may take place as a function of the user input. In addition, a graphic display of the permissible range may be displayed by a display unit.

In one embodiment, the representation REP of the examination object may include an image and/or a model of at least one anatomical structure and/or one medical object in the examination object. In this case, a spatial area that includes the at least one anatomical structure and/or the at least one medical object at least partially (e.g., in full) may be identified as the examination region to be mapped ID-ER. In addition, the representation REP may in each case have a candidate position relative to the mapped and/or modeled anatomical structures and/or the medical object. The initial isocenter may be identified at a candidate position within the examination region ER to be mapped ID-ITRAJ.

Alternatively, the initial isocenter may be identified based on a user input by the operating personnel, which is captured by the input unit.

In one embodiment, the initial isocenter and/or the at least one further isocenter may be identified based on geometric and/or anatomical features of the examination region to be mapped.

FIG. 2 shows a diagrammatic view of a further embodiment of a method for collision avoidance when positioning a medical imaging device and a patient positioning apparatus. After repositioning RPOS of the imaging device and/or the patient positioning apparatus, image data BD of the examination region may be acquired by the imaging device ACQ-BD. In addition, by reconstruction from the image data BD, a result data set ED including 3D mapping of the examination region may be provided RECO-ED.

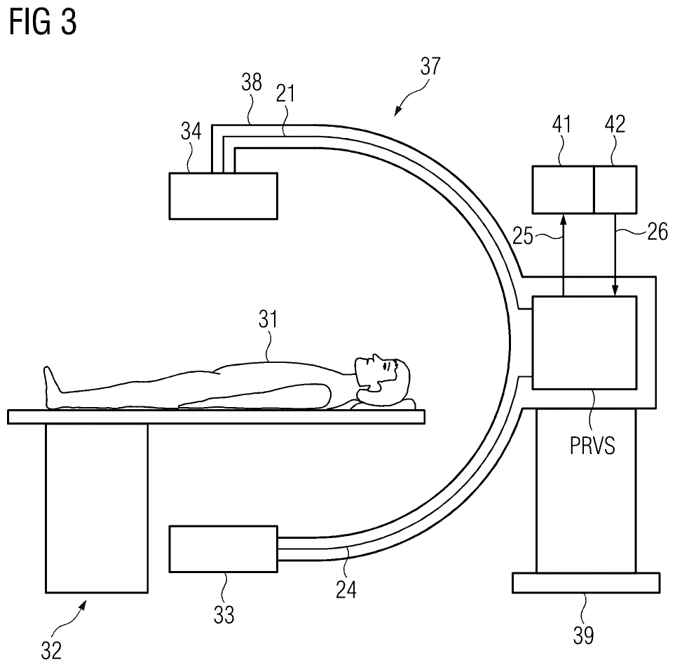

FIG. 3 shows a diagrammatic view of one embodiment of a system including a medical imaging device and a patient positioning apparatus 32. As an example of a medical imaging device, FIG. 3 shows a medical C-arm X-ray device 37 including a processing unit PRVS. The medical C-arm X-ray device 37 may have a detector 34 (e.g., an X-ray detector) and a source 33 (e.g., an X-ray source) that are arranged in a defined arrangement on a C-arm 38. The C-arm 38 of the C-arm X-ray device 37 may be mounted in a mobile manner around one or more axes. To acquire image data of the examination object 31 positioned on the patient positioning apparatus 32, the processing unit PRVS may send a signal 24 to the X-ray source 33. Thereupon, the X-ray source 33 may emit an X-ray beam. Upon impingement of the X-ray beam, after interaction with the examination object 31, on a surface of the detector 34, the detector 34 may send a signal 21 to the processing unit PRVS. The processing unit PRVS may capture the image data based on the signal 21.

Further, the system may have an input unit 42 (e.g., a keyboard) and a display unit 41 (e.g., a monitor and/or a display and/or a projector). The input unit 42 may be integrated into the display unit 41 (e.g., in the case of a capacitive and/or resistive touchscreen). The input unit 42 may be configured to capture the user input. The input unit 42 may, for example, send a signal 26 to the processing unit PRVS for this purpose. In an integrated embodiment of the input unit 42 and the display unit 41, with regard to a graphic display, the user input may be captured on the display unit 41.

The processing unit PRVS may be configured to identify the initial isocenter based on the user input. Alternatively or in addition, the imaging device and/or the patient positioning apparatus 32 may be repositioned (e.g., controlled) as a function of the captured user input. For example, a movement parameter (e.g., a speed of movement and/or a movement direction and/or a movement trajectory) and/or a target position for the imaging device and/or the patient positioning apparatus 32 may be specified by the user input.

The display unit 41 may be configured to display a graphic display of the permissible range and/or the result data set ED. The processing unit PRVS may send a signal 25 to the display unit 41 for this purpose.

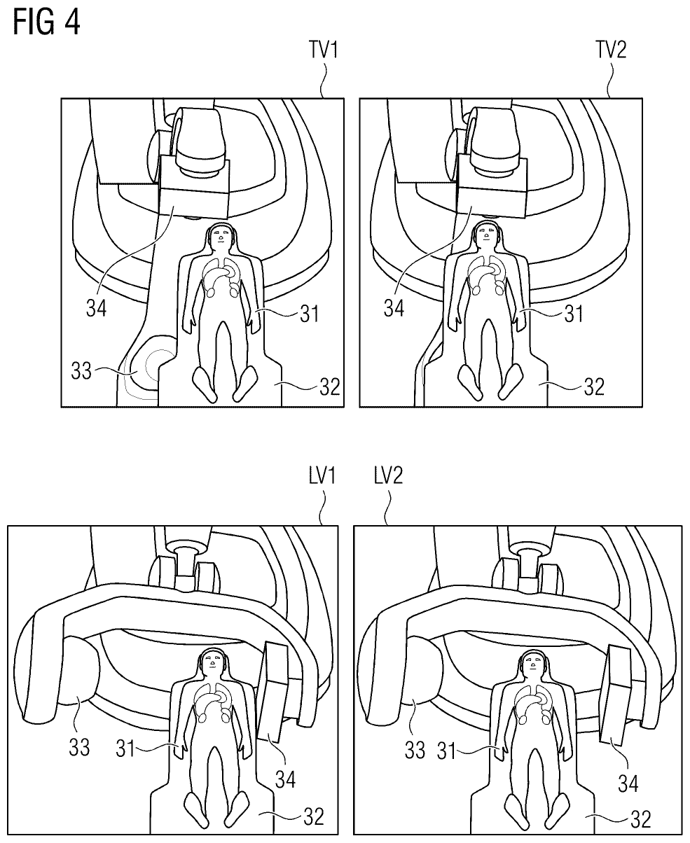

FIG. 4 is a diagrammatic view of a repositioning of the medical C-arm X-ray device 37 and the patient positioning apparatus 32 from various viewing perspectives. The representations TV1 and LV1 show a possible initial position that would lead to a collision, and the representations TV2 and LV2 show collision-free positioning of the patient positioning apparatus 32 and the C-arm X-ray device after repositioning. TV1 and TV2 show an anterior-posterior arrangement, and LV1 and LV show a lateral arrangement of the C-arm X-ray device 37 with regard to the examination object 31.

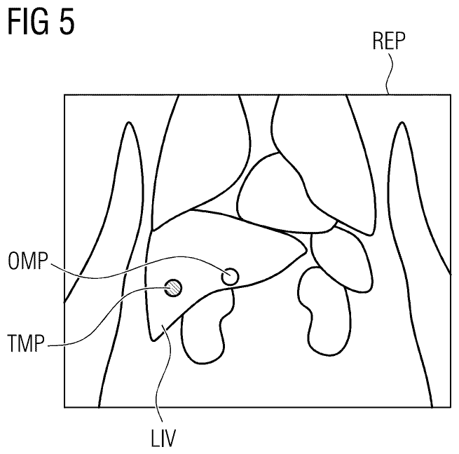

FIG. 5 shows a diagrammatic view of a representation REP of the examination object 31. The representation REP has a model of a liver LIV of the examination object 31, which may have been generated, for example, based on initial image data of the examination object 31 (e.g., using segmentation of anatomical structures). Based on anatomical features of the model of the liver LIV, TMP may be identified as the initial isocenter in tumor tissue to be mapped with a calculated initial acquisition trajectory around the initial isocenter ID-ITRAJ. This enables a 3D reconstruction of the tumor tissue using image data that may be acquired along the initial acquisition trajectory by the imaging device. In addition, the center of the organ (e.g., the organ focal point) may be identified as the at least one further isocenter with a further acquisition trajectory ID-FTRAJ.

The diagrammatic views contained in the figures described do not represent any scale or proportions.

The preceding methods described in detail and the apparatuses shown are only example embodiments that may be modified by a person skilled in the art in a variety of ways without departing from the scope of the invention. Further, the use of the indefinite articles “a” or “an” does not rule out the possibility that the features concerned may also be present multiple times. Likewise, the terms “unit” and “element” do not rule out the possibility that the components concerned include a plurality of interacting partial components that may also be distributed spatially if necessary.

The expression “based on” may be understood in the context of the present application, in particular, in the sense of the expression “using”. For example, a formulation according to which a first feature is generated (e.g., alternatively, ascertained, determined, etc.) based on a second feature does not rule out the possibility that the first feature may be generated (e.g., alternatively, ascertained, determined, etc.) based on a third feature.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for collision avoidance when positioning a medical imaging device and a patient positioning apparatus, the method comprising:
capturing a representation of an examination object that is arranged on a mobile patient positioning apparatus;
identifying an examination region to be mapped of the examination object based on a representation;
identifying an initial acquisition trajectory around an initial isocenter, wherein the initial acquisition trajectory enables a three-dimensional (3D) reconstruction of the examination region based on image data that is acquirable by the medical imaging device along the initial acquisition trajectory;
positioning the medical imaging device, the patient positioning apparatus, or the medical imaging device and the patient positioning apparatus such that an isocenter of the medical imaging device substantially coincides with the initial isocenter;
identifying at least one further acquisition trajectory around at least one further isocenter, wherein the at least one further acquisition trajectory enables a collision-free positioning of the imaging device and a 3D reconstruction of the examination region based on image data that is acquirable by the medical imaging device along the at least one further acquisition trajectory, and wherein the at least one further isocenter predefines a permissible range of relative positionings of the medical imaging device and the patient positioning apparatus; and
repositioning the medical imaging device, the patient positioning apparatus, or the medical imaging device and the patient positioning apparatus,
wherein the repositioning is limited to the permissible range of the relative positionings.

2. The method of claim 1, further comprising identifying a plurality of further acquisition trajectories around one further isocenter in each case, the plurality of further acquisition trajectories in each case enabling the collision-free positioning of the medical imaging device and the 3D reconstruction of the examination region based on image data that is acquirable by the medical imaging device along the respective further acquisition trajectory.

3. The method of claim 1, wherein during the repositioning of the medical imaging device, the patient positioning apparatus, or the medical imaging device and the patient positioning apparatus, a speed of movement of the medical imaging device, the patient positioning apparatus, or the medical imaging device and the patient positioning apparatus is reduced when approaching a border area of the permissible range of relative positionings.

4. The method of claim 1, wherein a user input by operating personnel is captured by an input unit, and
wherein the repositioning of the medical imaging device, the patient positioning apparatus, or the medical imaging device and the patient positioning apparatus takes place as a function of the user input.

5. The method of claim 1, wherein a graphic display of the permissible range is indicated by a display unit.

6. The method of claim 1, wherein the representation of the examination object has an image, a model, or an image and a model of at least one anatomical structure, a medical object in the examination object, or the at least one anatomical structure and the medical object in the examination object, and
wherein a spatial area that at least partially comprises the at least one anatomical structure, the medical object, or the at least one anatomical structure and the medical object is identified as the examination region to be mapped.

7. The method of claim 6, wherein the representation of the examination object has a candidate position in each case with regard to the at least one anatomical structure, the medical object to be mapped, modeled, or mapped and modeled, or a combination thereof, and
wherein the initial isocenter is identified at a candidate position within the examination region to be mapped.

8. The method of claim 1, wherein the initial isocenter is identified based on a user input by operating personnel using an input unit.

9. The method of claim 1, wherein the initial isocenter, the at least one further isocenter, or the initial isocenter and the at least one further isocenter are identified based on geometric, anatomical, or geometric and anatomical features of the examination region to be mapped.

10. The method of claim 1, wherein the medical imaging device comprises a medical X-ray device with an X-ray detector and an X-ray source that are arranged in a defined arrangement on an arcuate structure, and
wherein the initial acquisition trajectory and the at least one further acquisition trajectory specify a movement of the defined arrangement around the respective isocenter.

11. The method of claim 1, further comprising acquiring, by the medical imaging device, image data of the examination region after the repositioning of the medical imaging device, the patient positioning apparatus, or the medical imaging device and the patient positioning apparatus,
wherein a result data set having 3D mapping of the examination region by reconstruction is provided from the image data.

12. A system comprising:
a medical imaging device; and
a patient positioning apparatus,
wherein the medical imaging device and the patient positioning apparatus are configured for collision avoidance when positioning a medical imaging device and a patient positioning apparatus, the collision avoidance comprising:
capture of a representation of an examination object that is arranged on a mobile patient positioning apparatus;
identification of an examination region to be mapped of the examination object based on a representation;
identification of an initial acquisition trajectory around an initial isocenter, wherein the initial acquisition trajectory enables a three-dimensional (3D) reconstruction of the examination region based on image data that is acquirable by the medical imaging device along the initial acquisition trajectory;
position of the medical imaging device, the patient positioning apparatus, or the medical imaging device and the patient positioning apparatus such that an isocenter of the medical imaging device substantially coincides with the initial isocenter;
identification of at least one further acquisition trajectory around at least one further isocenter, wherein the at least one further acquisition trajectory enables a collision-free positioning of the imaging device and a 3D reconstruction of the examination region based on image data that is acquirable by the medical imaging device along the at least one further acquisition trajectory, and wherein the at least one further isocenter predefines a permissible range of relative positionings of the medical imaging device and the patient positioning apparatus; and
reposition of the medical imaging device, the patient positioning apparatus, or the medical imaging device and the patient positioning apparatus,
wherein the reposition is limited to the permissible range of the relative positionings.

13. In a non-transitory computer-readable storage medium that stores instructions executable by one or more processors for collision avoidance when positioning a medical imaging device and a patient positioning apparatus, the instructions comprising:
capturing a representation of an examination object that is arranged on a mobile patient positioning apparatus;
identifying an examination region to be mapped of the examination object based on a representation;
identifying an initial acquisition trajectory around an initial isocenter, wherein the initial acquisition trajectory enables a three-dimensional (3D) reconstruction of the examination region based on image data that is acquirable by the medical imaging device along the initial acquisition trajectory;
positioning the medical imaging device, the patient positioning apparatus, or the medical imaging device and the patient positioning apparatus such that an isocenter of the medical imaging device substantially coincides with the initial isocenter;
identifying at least one further acquisition trajectory around at least one further isocenter, wherein the at least one further acquisition trajectory enables a collision-free positioning of the imaging device and a 3D reconstruction of the examination region based on image data that is acquirable by the medical imaging device along the at least one further acquisition trajectory, and wherein the at least one further isocenter predefines a permissible range of relative positionings of the medical imaging device and the patient positioning apparatus;
repositioning the medical imaging device, the patient positioning apparatus, or the medical imaging device and the patient positioning apparatus,
wherein the repositioning is limited to the permissible range of the relative positionings.

14. The non-transitory computer-readable storage medium of claim 13, wherein the instructions further comprise identifying a plurality of further acquisition trajectories around one further isocenter in each case, the plurality of further acquisition trajectories in each case enabling the collision-free positioning of the medical imaging device and the 3D reconstruction of the examination region based on image data that is acquirable by the medical imaging device along the respective further acquisition trajectory.

15. The non-transitory computer-readable storage medium of claim 13, wherein during the repositioning of the medical imaging device, the patient positioning apparatus, or the medical imaging device and the patient positioning apparatus, a speed of movement of the medical imaging device, the patient positioning apparatus, or the medical imaging device and the patient positioning apparatus is reduced when approaching a border area of the permissible range of relative positionings.

16. The non-transitory computer-readable storage medium of claim 13, wherein a user input by operating personnel is captured by an input unit, and
wherein the repositioning of the medical imaging device, the patient positioning apparatus, or the medical imaging device and the patient positioning apparatus takes place as a function of the user input.

17. The non-transitory computer-readable storage medium of claim 13, wherein a graphic display of the permissible range is indicated by a display unit.

18. The non-transitory computer-readable storage medium of claim 13, wherein the representation of the examination object has an image, a model, or an image and a model of at least one anatomical structure, a medical object in the examination object, or the at least one anatomical structure and the medical object in the examination object, and
wherein a spatial area that at least partially comprises the at least one anatomical structure, the medical object, or the at least one anatomical structure and the medical object is identified as the examination region to be mapped.
